(19) United States
(12) Patent Application Publication
(10) Pub. No.: US 2021/0393547 A1
(43) Pub. Date: Dec. 23, 2021

(54) USE OF EMODIN IN TREATING RETINAL ISCHEMIA OR A DISEASE, CONDITION, OR DISORDER ASSOCIATED WITH RETINAL ISCHEMIA

(71) Applicant: Hsiao-Ming Chao, Taipei City (TW)

(72) Inventor: Hsiao-Ming Chao, Taipei City (TW)

(21) Appl. No.: 16/907,310

(22) Filed: Jun. 22, 2020

(51) Int. Cl. A61K 31/122; A61P 27/02

(52) U.S. Cl. CPC A61K 31/122; A61P 27/02

(57) ABSTRACT

A method for treating a subject suffering from retinal ischemia, or a disease, condition, or disorder associated with retinal ischemia, comprising administering to the subject a therapeutically effective amount of emodin or a composition comprising emodin.

### FIELD OF THE INVENTION

The present invention relates to use of emodin in the treatment of retinal ischemia. The invention relates more particularly to the use of a composition comprising emodin intended for the treatment and/or the prevention of a retinal ischemia and disease, condition, or disorder associated with retinal ischemia.

### BACKGROUND OF THE INVENTION

Clinical Relevance to the Retinal Ischemic Model

Retinal vascular occlusion (central/branch retinal artery/vein occlusion), glaucomatous optic neuropathy, proliferative diabetic retinopathy (DBR), and neovascular age-related macular degeneration (AMD), as well as retinal developmental anomalies are conditions that have been associated with retinal ischemia. Clinically, retinal ischemia is detected when there are alterations in the b-wave of the electroretinogram (ERG), optical coherence tomography (OCT)-proved retinal thinning, and/or alterations in visual field due to the death of inner retinal neurons (e.g. retinal ganglion cells (RGCs)). These diseases affect millions of people worldwide, and therefore the management of retina ischemia is very important. A model involving the induction of retinal ischemia was therefore established that involved increasing intraocular pressure (IOP). Using this approach, an investigation into novel therapeutic approaches related to various signaling pathways would be a useful way to find an appropriate agent against retinal ischemia.

Exploring β-Catenin and VEGF

Studies have shown that β-catenin signaling pathway activates T-cell factor-4 (TCF-4) and promotes cell proliferation under normoxic condition. However, in hypoxia, β-catenin promotes the expression of hypoxia inducible factor-1α (HIF-1α), which is widely known to subsequently elevate the level of vascular endothelial growth factor (VEGF). This event in turn arrests the cell cycle as an adaptation to hypoxia. Additionally, the inhibition of protein expression of β-catenin and/or VEGF has been reported to prevent an ischemia induced increase in vascular permeability that is related to consequential neovascularization, ocular hemorrhage and/or cystoid macular edema.

Emodin

Emodin (6-methyl-1,3,8-trihydroxyanthraquinone) is a chemical compound that can be isolated from rhubarb, buckthorn, and Japanese knotweed. Emodin has shown potential to inhibit inflammation in various settings. For instance, emodin has been shown to attenuate the severity of experimental disease models including arthritis, liver damage, atherosclerosis, myocardial ischemia, and cancer.

### SUMMARY OF THE INVENTION

The present invention relates to a method for treating a subject suffering from retinal ischemia, or a disease, condition, or disorder associated with retinal ischemia, comprising administering to said subject a therapeutically effective amount of emodin or a composition comprising emodin.

### BRIEF DESCRIPTION OF THE DRAWINGS

FIG. 1 shows that the viability of the cultured RGC-5 cell line is quantitatively analyzed using MTT assay. The value in each group is the ratio of the viability of the cultured RGC-5 cells relative to that of the control group, which is set as 100%. This study includes four groups, namely control group (normal; RGC-5 cells which were cultured in culture medium containing vehicle), Pre-OGD vehicle group (1 hour of pre-OGD treatment with vehicle), Pre-OGD Emo 0.25 μM group (1 hour of pre-OGD treatment with 0.25 μM emodin), and Pre-OGD Emo 0.5 μM group (1 hour of pre-OGD treatment with 0.5 μM Emodin). *** identifies that the normal control group significantly differs (P<0.001) from the Pre-OGD vehicle group. † designates a significant difference (P=0.04) between the Pre-OGD vehicle group and the Pre-OGD Emo 0.5 μM group. The results are mean±SE (n=5˜6). Abbreviations: RGC-5, retinal ganglion cell-5; MTT, 3-(4,5-dimethylthiazol-2-yl)-2,5-diphenyltetrazolium bromide; OGD, oxygen glucose deprivation; Emo, Emodin.

FIG. 2 shows summary of ERG b-wave measurement. FIG. 2a shows electroretinogram (ERG) b-wave amplitude. After ischemia/reperfusion (IR), a substantially reduced ERG b-wave amplitude is demonstrated in the ischemic retina pre-administered with intravitreous vehicle (Vehicle+IR) as compared with the control retina (Sham). In a dose-dependent way, pre-administered intravitreous emodin [4, 10 and 20 μM, i.e. Emo4+IR, Emo10+IR and Emo20+IR (n=4)], but not vehicle, ameliorates the ischemia induced reduction in ERG b-wave amplitude. Post-ischemic intravitreous injection of 20 μM emodin (IR+Emo20) also has an anti-ischemic effect. FIG. 2b shows the analysis of ERG b-wave ratios revealing the efficacy of pre-ischemic and post-ischemic administration of emodin on the ischemic retina. As compared to the ERG b-wave ratio of the Control (Sham=0.82±0.14: normalized to 1, n=7), a significant (***; P<0.001) reduction in that of the Vehicle+IR group is revealed. In contrast to the Vehicle+IR group (0.04±0.01, n=8), pre-ischemic intravitreous injection of emodin dose-responsively (Emo4+IR=0.08±0.07, n=8; Emo10+IR=0.64±0.28, n=4; then, Emo20+IR=0.99±0.18, n=4) and significantly (†††; P<0.001; Emo10+IR; Emo20+IR) attenuate ischemic insult. Post-ischemic intravitreous injection of 20 μM emodin (IR+Emo20=0.24±0.09, n=9) has a significant (†††; P<0.001) anti-ischemic effect, too. Data are mean±S.E.M. of the number of animals shown in the parenthesis. Abbreviations for group names are provided in Table 3.

FIG. 3 shows calculation of thickness of cresyl violet stained retinas. FIGS. 3a to 3f show cresyl violet stained retinal sections with the same eccentricity. Micrographs show the whole (top row) or inner retina (bottom row) thickness (μm) of different groups. FIGS. 3a and 3b, 3g and 3h show that as compared with the retinal thickness of the control group (Sham: whole=186.50±1.43; inner=79.90±2.06), a substantial reduction in the whole or inner retina thickness is observed in that of the Vehicle+IR group (whole=71.80±1.08; inner=20.97±0.85). FIGS. 3c to 3e, 3g and 3h show that pre-ischemic intravitreous injection of emodin dose-responsively (least effect at 4 μM; Emo4+IR: whole=87.40±0.60, inner=38.60±1.01; then, 10 μM; greatest effect at μM) and in a significant way (Emo10+IR: whole=153.20±1.48, inner=70.05±0.60; Emo20+IR: whole: 170.10±0.10; 70.65±2.06) attenuate ischemia induced reduction in the whole and inner retina thickness. FIGS. 3f, 3g and 3h show that post-ischemic intravitreous injection of 20 μM emodin also significantly alleviates ischemia reduced whole and inner retina thickness (IR+Emo20: whole=125.45±1.68, inner=69.65±0.68). FIGS. 3g and 3h show quantitative analysis of the whole or inner retina thickness. *** or †††/†† indicates a significant (P<0.001 or P<0.001/P<0.01) difference from the Sham or Vehicle+IR group. Abbreviations: IR, ischemia plus reperfusion; ONL, outer nuclear layer; OPL, outer plexiform layer; INL, inner nuclear layer; IPL, inner plexiform layer; GCL, ganglion cell layer. Scale bar is 50 μm. The results are mean±standard error. Abbreviations for group names are provided in Table 4.

FIG. 4 shows fluorogold retrograde labelling. Micrographs show the density of retinal ganglion cell (RGC) at various groups. FIGS. 4a and 4e show that at the Sham group, the highest density of RGCs is observed 5323.53±215.6/0.17 mm². FIGS. 4b and 4e show that at the Vehicle+IR group, an obvious reduction in the cell density is demonstrated. FIGS. 4c, 4d and 4e show pre-ischemic intravitreous injection of emodin 10 and 20 μM (Emo10+IR; Emo20+IR) in a dose dependent manner and significantly increased the cell density; FIG. 4e shows quantitative analysis of the RGC density. *** or ††/††† denotes a significant (P<0.001 or P<0.01/P<0.001) difference from the Sham or Vehicle+IR group. Emodin dose-dependently and significantly counteracts the ischemia induced reduction in the RGC density. The results are mean±standard error of the amount of animals demonstrated in the parenthesis. Scale bar is 50 μm. Abbreviations for group names are provided in Table 5.

FIG. 5 shows western blot assay. FIG. 5a: blotting images of β-catenin, vascular endothelium growth factor (VEGF) and β-actin protein. Lane 1 is from a sham retina (Control); Lane 2 is the vehicle-pretreated ischemic retina (Vehicle+IR); Lanes 3 and 4 are from retinas that were subjected to IR and were pretreated with 10 μM (Emo10+IR) and 20 μM emodin (Emo20+IR). FIGS. 5b and 5c: the bar chart analyzing the ratio of β-catenin/VEGF to the house-keeping protein β-actin. The ratio of the Sham group is normalized to 1. ***/* represents an extremely significant (P<0.001) or a significant (P<0.05) difference as compared to the Sham group. † represents a significant (P=0.02/0.03) difference when compared to the Vehicle+IR group. As compared to the sham group, a significant increase in β-catenin/VEGF protein levels is observed after an ischemic insult and pre-ischemic application of vehicle (Vehicle+IR=1.64±0.14/7.67±2.57). In contrast, pre-ischemic application of emodin dose-dependently and significantly (P=0.02/0.03 at 20 μM; Emo20+IR=1.00±0.19/1.23±0.44) inhibit ischemia induced increase in β-catenin/VEGF protein levels. The values are mean±S.E of the number of animals illustrated in the parenthesis. Abbreviations are listed as follows. IR: ischemia plus reperfusion; Pre-ischemic emodin 10 μM followed by IR (Emo10+IR); Pre-ischemic emodin 20 μM followed by IR (Emo20+IR). Abbreviations for group names are provided in Table 6.

### DETAILED DESCRIPTION OF THE INVENTION

In the present invention, an animal model is used to investigate the protective effects of emodin against retinal ischemia. Furthermore, an investigation is made into how the levels of β-catenin might be regulated in the ischemic retina treated with emodin.

The present invention has demonstrated that raised IOP induced retinal ischemic injury is able to be alleviated by pre-ischemic and/or post-ischemic emodin application. The alterations induced by ischemia are monitored using electroretinography, histopathology (cresyl violet stained retinal layer thickness), retrograde fluorogold immunolabled RGCs, and measurement of β-catenin and VEGF. In the MTT cell viability assay, pre-OGD (oxygen glucose deprivation) administration of 0.5 μM emodin (an equivalent intravitreous injection dose of 20 μM in a rat) significantly attenuates the OGD induced cellular injury. Furthermore, in the animal study, pre-ischemic intravitreous injection of 20 μM emodin significantly attenuates the retinal ischemia induced reduction in ERG b-wave amplitude. The post-ischemic intravitreous injection group also significantly alleviates this b-wave reduction. This protective effect is also present when cresyl violet stained retinal thickness and/or retrograde fluorogold immunolabeled RGC density are used. Additionally, β-catenin/VEGF protein levels have been proven to be significantly increased after retinal ischemic injury. These elevations are significantly blunted by pre-treatment with emodin. The above findings imply that emodin has a protective effect against retinal ischemia injured neurons such as RGCs via the downregulation of ischemia induced β-catenin/VEGF protein upregulation.

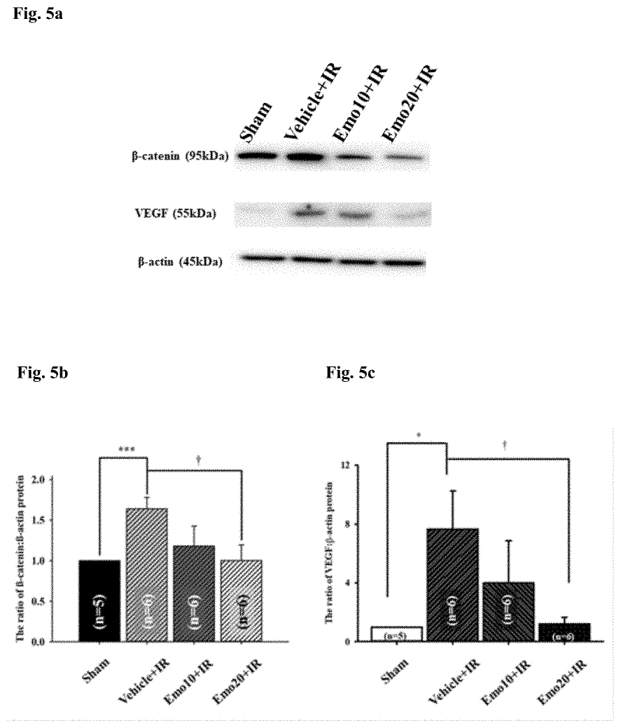

Under ischemic conditions (hypoxia), upregulated HIF-1α protein competes with TCF-4 to bind with cellular β-catenin. Once bound to HIF-1α, β-catenin will rapidly switch roles from co-activating TCF-4 to stimulating HIF-1α mediated transcription. An increase in HIF-1α mediated transcription under ischemic conditions leads to downstream VEGF upregulation with a range of possible sequelae including macular edema and/or ocular bleeding. The present invention indicates that the emodin has an ameliorating effect on ischemic damage done to the retinas, while at the same time there is a dose-dependent and significant (at 20 μM) downregulation of ischemia induced β-catenin/VEGF overexpression. Therefore, the protective mechanisms of emodin would seem to involve a decrease in β-catenin/VEGF protein levels together with a decrease in the aforementioned β-catenin coactivated HIF-1α mediated transcription and a consequent reduction in VEGF concentrations. All these findings and the present protein analysis (FIG. 5) strongly support that emodin has an anti-ischemic and protective effect via an inhibition of the ischemia induced overexpression of β-catenin and VEGF (FIG. 5). This is of major clinical importance, because it can point the way of managing clinically proved complications (e.g. macular edema) due to retinal ischemic disorders. These include retinal vascular occlusion, proliferative diabetic retinopathy, neovascular age related maculopathy and, possibly, retinal developmental diseases (such as familial exudative vitreoretinopathy (FEVR), Coats' disease, persistent hyperplastic primary vitreous (PHPV), Norrie disease or retinopathy of prematurity (ROP).

Therefore, the present invention provides a method for treating a subject suffering from retinal ischemia, or a disease, condition, or disorder associated with retinal ischemia, comprising administering to said subject a therapeutically effective amount of emodin or a composition comprising emodin. The therapeutically effective amount of administered emodin can be from 2 to 30 μM. In an embodiment, the therapeutically effective amount of administered emodin can be from 4 to 20 μM. In an embodiment, the therapeutically effective amount of administered emodin is selected from the group consisting of 4, 10 and 20 μM. In an embodiment, the emodin is administered by intravitreous injection. In another embodiment, the emodin is administered by oral administration.

In an embodiment, the disease, condition, or disorder associated with retinal ischemia comprises retinal vascular occlusion, glaucomatous optic neuropathy, proliferative diabetic retinopathy, neovascular age-related macular degeneration, familial exudative vitreoretinopathy (FEVR), Coats' disease, persistent hyperplastic primary vitreous (PHPV), Norrie disease or retinopathy of prematurity (ROP). In a preferred embodiment, the disease, condition, or disorder associated with retinal ischemia comprises FEVR, Coat's disease, PHPV, Norrie disease and retinopathy of prematurity.

In an embodiment, the subject is a mammal, in a preferred embodiment, the subject is a human.

In an embodiment, the above method further comprises administering to said subject a pharmaceutically acceptable adjuvant, vehicle, or carrier.

In an embodiment, the composition treats retinal ischemia, or the disease, condition, or disorder associated with retinal ischemia by inhibiting the overexpression of β-catenin and VEGF induced by retinal ischemia.

The present invention also provides a method for treating or lessening the severity of a subject suffering from a disease, condition, or disorder in which modulation of β-catenin is beneficial, comprising administering to said subject a therapeutically-effective amount of emodin or a composition comprising emodin. The therapeutically effective amount of administered emodin can be from 2 to 30 μM. In an embodiment, the therapeutically effective amount of administered emodin can be from 4 to 20 μM. In an embodiment, the therapeutically effective amount of administered emodin is selected from the group consisting of 4, 10 and 20 μM. In an embodiment, the emodin is administered by intravitreous injection. In another embodiment, the emodin is administered by oral administration.

In an embodiment, the subject is a mammal, in a preferred embodiment, the subject is a human.

In an embodiment, the above method further comprises administering to said subject a pharmaceutically acceptable adjuvant, vehicle, or carrier.

The present invention further provides a method for protecting cells from injury caused by retinal ischemia in a subject suffering from retinal ischemia, comprising administering to said subject an effective amount of emodin or a composition comprising emodin, wherein the cells are selected from the group consisting of bipolar cells, Müller cells, and cholinergic amacrine cells. The therapeutically effective amount of administered emodin can be from 2 to 30 μM. In an embodiment, the therapeutically effective amount of administered emodin can be from 4 to 20 μM. In an embodiment, the therapeutically effective amount of administered emodin is selected from the group consisting of 4, 10 and 20 μM. In an embodiment, the emodin is administered by intravitreous injection. In another embodiment, the emodin is administered by oral administration.

In an embodiment, the composition protects cells from injury caused by retinal ischemia in the subject in need thereof by inhibiting the overexpression of β-catenin and VEGF induced by retinal ischemia.

### Abbreviations

ERG: Electroretinogram; OCT: optical coherence tomography; RGC: retinal ganglion cells; IOP; intraocular pressure; HIOP: High intraocular pressure; TCF-4: T-cell factor-4; HIF-1α: Hypoxia-inducible factor-1α; OGD: oxygen glucose deprivation; MTT: 3-(4,5-dimethylthiazol-2-yl)-2,5-diphenyltetrazolium bromide; IR: ischemia plus reperfusion; Emo: emodin; ARRIVE: Animal Research Reporting of In Vivo Experiments; ILM: Internal limiting membrane; RPE: retinal pigment epithelium; INL: Inner nuclear layer; SDS-PAGE: sodium dodecyl sulfate polyacrylamide gel electrophoresis; PVDF: polyvinylidene fluoride; P: Probability; VEGF: vascular endothelial growth factor.

### EXAMPLES

The examples below are non-limiting and are merely representative of various aspects and features of the present invention.

### Example 1

Materials and Methods

Cellular Experiments

Oxygen Glucose Deprivation

RGC-5 cells have been reported to be not transformed rat RGCs, but rather are mouse retinal neuronal precursor cells. OGD was established by incubating RGC-5 cells in Dulbecco's modified Eagle medium (DMEM; Thermo Fisher Scientific Inc.) without glucose at 37° C. under ischemia simulation conditions, namely 1% O₂ (measured using a Penguin Incubator; Astec Company, Kukuoka, Japan), 94% N₂ and 5% CO₂. Various experimental groups (Table 1) were investigated. These were cells treated with: (i) culture medium containing vehicle (control group), (ii) 1 hour of pre-OGD treatment with vehicle (Pre-OGD vehicle group), (iii) 1 hour of pre-OGD treatment with 0.25 μM emodin (Pre-OGD Emo 0.25 μM group), (iv) 1 hour of pre-OGD treatment with 0.5 μM emodin (Pre-OGD Emo 0.5 μM group). Following 24 hours of OGD, the cell cultures were transferred to new DMEM for another 1 day. MTT assays to assess cell viability were then carried out.

MTT Assay

Mitochondrial nicotinamide adenine dinucleotide phosphate dependent oxidoreductases are able to reduce MTT to produce formazan and thus increased levels of dark purple formazan that are associated with greater cell viability. MTT (0.5 mg/mL; Sigma-Aldrich) was added to 100 μL of cells in each well of 96-well plates for 3 hours at 37° C. After reduction of the MTT, the formazan was dissolved by the addition of 100 μL of DMSO. After shaking, the optical density (OD) of the dissolved formazan was measured using an ELISA plate reader (Synergy H1 Multi-Mode Reader BioTek Instruments) at 562 nm. Cell viability was calculated as the change in OD value compared to the untreated control (100%).

TABLE 1
Quantitative analysis of the viability of the
cultured RGC-5 cell line utilizing MTT assay.

| | Group (n = 5~6) | | | |
|---|---|---|---|---|
| | Con- | Pre-OGD | Pre-OGD | Pre-OGD |
| | trol | vehicle | Emo 0.25 μM | Emo 0.5 μM |
| Cell | 100 | 38.30 ± 2.51%*** | 43.81 ± 3.75% | 47.52 ± 3.99%† |
| viability | | | | |
| (% of | | | | |
| control) | | | | |

The value in each group was the ratio of the viability of the cultured RGC-5 cells relative to that of the control group, which was set as 100%. This study include 4 groups including control group (normal; RGC-5 cells that were incubated in culture medium containing vehicle), Pre-OGD vehicle group (1 hour of pre-OGD administration of vehicle before the OGD condition), Pre-OGD Emo 0.25 μM group (1 hour pre-OGD administration of 0.25 μM emodin before the OGD condition), and Pre-OGD Emo 0.5 μM group (1 hour pre-OGD administration of 0.5 μM Emodin). *** denotes an extremely significant difference (P<0.001) between the normal control and the OGD group. † denotes a significant difference (P=0.04) between the OGD group and the pre-OGD application of emodin 0.5 μM group. The results are mean±SE (n=5˜6). Abbreviations: RGC-5, retinal ganglion cell-5; MTT, 3-(4,5-dimethylthiazol-2-yl)-2,5-diphenyltetrazolium bromide; OGD, oxygen glucose deprivation; Emo, Emodin.

Animal Experiments

Animal Use

The animals were Wistar rats purchased from BioLasco, Taipei. Upon arrival, the Wistar rats were 6 weeks old and weighed 175˜250 g. They were raised in groups of less than six rats inside a large plastic cage (Shineteh Instruments Co., Ltd., Taipei). The humidity and temperature were controlled to be within the ranges 40% to 60% and 21±2° C., respectively. Rats were assigned randomly to the various experimental groups. The number of the rats used in this invention was 120 (=99+21; 99=60+16+23; as shown in Table 2). These include animals (n=21) that died during the procedures; these occurred during retinal ischemic induction (n=9), ERG detection (n=4), and fluorogold retrograde RGC labelling (n=8). After ERG recordings had been performed, all surviving rats (n=99) underwent the following procedures, namely cresyl violet staining, fluorogold labelling and Western blot assays. The surviving animals, each of which had been subjected to a sham procedure, IR with pre-administered vehicle, or IR with pre-/post-administered emodin (Emo) formed the following groups: Sham (n=19), Vehicle+IR (n=20), Emo4+IR (n=10), Emo10+IR (n=20), Emo20+IR (n=20) and IR+Emo20 (n=10). The animal experimental procedures were performed in a way that adhered to the Animal Research: Reporting of In Vivo Experiments (ARRIVE) guidelines.

TABLE 2

| Group/Method | CV† | FG† | WB† | Total* |
|---|---|---|---|---|
| Sham | 10 | 4 | 5 | 19 |
| Vehicle + IR | 10 | 4 | 6 | 20 |
| Emo4 + IR | 10 | — | — | 10 |
| Emo10 + IR | 10 | 4 | 6 | 20 |
| Emo20 + IR | 10 | 4 | 6 | 20 |
| IR + Emo20 | 10 | — | — | 10 |
| Subtotal No. | 60 | 16 | 23 | 99 |

*The number of the rats used in this invention was 120 (=99 + 21; 99 = 60 + 16 + 23), including animals (n = 21) that died during the following procedures, namely in the HIOP (n = 9), ERG (n = 4), and FG (n = 8).
†After ERG recordings, all recorded rats (n = 99) were preserved for the following procedures, namely CV, FG, and WB.
Abbreviations:
Sham, normal control;
IR, ischemia/reperfusion;
Emo4, Emodin 4 μM;
Emo10, Emodin 10 μM;
Emo20, Emodin 20 μM;
CV, Cresyl violet stain;
FG, fluorogold;
WB, western blot;
ERG, electroretinogram.

Animal Anesthesia and Euthanasia

The Wistar rats were anesthetized using a combination of intraperitoneal ketamine (Pfizer; 100 mg/kg) and xylazine (Sigma-Aldrich; 5 mg/kg) to bring about pain relief and sedation. ERG recordings, fluorogold intracranial injections and intravitreous injections of defined agents were performed. To minimize pain and to humanely sacrifice the Wistar rats, a final intraperitoneal injection of at least 140 mg/kg of sodium pentobarbital (SCI Pharmtech) was given to each subject.

Administration of Drug

Emodin was purchased from Sigma-Aldrich (E7881; 90%; storage at 2-8° C.; from Frangula bark; St. Louis, Mo., United States). Drug administration for different procedures, namely ERG, fluorogold RGC labelling, cresyl violet retinal staining and Western blot assays, were described in Tables 3 to 6. Vehicle or emodin (5 μL) was administered via an intravitreous injection route so that these agents were then able to be directly diffused to the target. Emodin was dissolved in vehicle (DMSO:distilled water=1:3). The defined final cellular concentrations of administered drug were 0.25 or 0.5 μM, which were achieved after 40 times dilution from the stock concentrations, namely 10 or 20 μM, based on an arbitrary definition of the vitreous volume as 200 μL. To test a wider range of dose-response, 4 μM of emodin was thus added in the animal experiments. The Vehicle+IR and Emo+IR groups (pre-ischemic administration of emodin 4, 10 or 20 μM) were subjected to following the experimental procedures; these were initial pre-ischemic intravitreous injection (vehicle or emodin), retinal ischemia induction 1 day later, and sacrifice of the animal on the following day to allow the various post-mortem procedures except ERG. In the post-ischemic treatment group, in order to decrease the number of animals used, the present invention only used a single emodin concentration of 20 μM. Furthermore, intravitreous emodin was given 1 day after initial retinal ischemia induction and animals were sacrificed the following day to allow the performance of various post-mortem procedures as mentioned above.

TABLE 3
Group names and conditions at various
groups in the ERG b-wave analysis

| | Definition of conditions in which the retina |
|---|---|
| Group name | has been treated |
| Sham (n = 7; control) | Sham procedure |
| Vehicle + IR (n = 8) | Pre-ischemic intravitreous injection of vehicle |
| | followed by IR |
| Emo4 + IR (n = 8) | Pre-ischemic intravitreous injection of 4 μM |
| | emodin followed by IR |
| Emo10 + IR (n = 4) | Pre-ischemic intravitreous injection of 10 μM |
| | emodin followed by IR |
| Emo20 + IR (n = 4) | Pre-ischemic intravitreous injection of 20 μM |
| | emodin followed by IR |
| IR + Emo20 (n = 9) | IR followed by post-ischemic intravitreous |
| | injection of 20 μM emodin |

Abbreviations:
ERG, electroretinogram;
IR, ischemia/reperfusion

TABLE 4
Group names and conditions used at various groups evaluating
the cresyl violet stained retinal thickness

| | Definition of conditions in which the retina |
|---|---|
| Group name | has been treated |
| Sham (n = 10; control) | Sham procedure |
| Vehicle + IR (n = 10) | Pre-ischemic intravitreous injection of vehicle |
| | followed by IR |
| Emo4 + IR (n = 10) | Pre-ischemic intravitreous injection of 4 μM |
| | emodin followed by IR |
| Emo10 + IR (n = 10) | Pre-ischemic intravitreous injection of 10 μM |
| | emodin followed by IR |
| Emo20 + IR (n = 10) | Pre-ischemic intravitreous injection of 20 μM |
| | emodin followed by IR |
| IR + Emo20 (n = 10) | IR followed by post-ischemic intravitreous |
| | injection of 20 μM emodin |

TABLE 5
Group names and conditions at various groups
analyzing retrograde fluorogold labeled RGCs

| | Definition of conditions in which the retina |
|---|---|
| Group name | has been treated |
| Sham (n = 4; control) | Sham procedure |
| Vehicle + IR (n = 4) | Pre-ischemic intravitreous injection of vehicle |
| | followed by IR |
| Emo10 + IR (n = 4) | Pre-ischemic intravitreous injection of 10 μM |
| | emodin followed by IR |
| Emo20 + IR (n = 4) | Pre-ischemic intravitreous injection of 20 μM |
| | emodin followed by IR |

TABLE 6
Group names and conditions at various
groups in the western blotting analysis

| | Definition of conditions in which the retina |
|---|---|
| Group name | has been treated |
| Sham (n = 6; control) | Sham procedure |
| Vehicle + IR (n = 6) | Pre-ischemic intravitreous injection of vehicle |
| | followed by IR |
| Emo10 + IR (n = 6) | Pre-ischemic intravitreous injection of 10 μM |
| | emodin followed by IR |
| Emo20 + IR (n = 6) | Pre-ischemic intravitreous injection of 20 μM |
| | emodin followed by IR |

Induction of Retinal Ischemia

The IOP procedure used raised the IOP and was designed to cause retinal ischemia in Wistar rats and simulate ischemic retinal disorders. The rats were anesthetized and constrained with a sterotaxic frame. A 30-gauge needle connected to an elevated saline bottle was attached to anterior chamber of the rat's eye. The pressure exerted by the saline inside the bottle was controlled to 120 mmHg. This procedure was conducted for 1 hour. Successful retinal ischemia induction was confirmed by observing that the whitening of the retina took place. Throughout the experiments, the animals were kept on heat mats to maintain their temperature. The sham procedure involved keeping the pressure in saline bottle 0 mmHg.

ERG Recording

Animals were given anesthesia prior to the flash ERG measurements. For the Sham or pre-ischemic treatment group (intravitreous injection of emodin or vehicle 1 day before ischemia; Emodin+IR or Vehicle+IR), flash ERG responses were measured before the sham procedure, IR procedures or any drug administration (day 0), and one day after the sham procedure or IR procedure with pre-ischemic intravitreous injection. In the post-ischemic treatment group, ERG recordings were collected pre-ischemia (day 0), and post-ischemia (at timepoints: 1 day after ischemia; 1 day after post-ischemic intravitreous injection of emodin). Dark adaption and pupil dilation using 1% tropicamide and 2.5% phenylephrine were carried out 8 hours before the ERG measurement. The ERG recording machinery was purchased from the Grass-Telefactor Company (AstroNova, QC, Canada) and included a stimulator (PS22), a regulated power supply (RPS107), and an amplifier (P511). A strobe light (0.5 Hz), which acted as the source of stimulus, was placed 2 cm directly in front of the rat's eye. Fifteen consecutive measurements (10 kHz) were retrieved every two seconds. The amplitudes were calculated to obtain an average. To make comparisons between the various groups, the ratios of the b-wave amplitude of one eye (sham procedure or ischemic insult with defined agents) to that of the untreated fellow normal eye were analyzed.

Cresyl Violet Staining

After the aforementioned sacrifice of rats, the animals underwent intracardial perfusion with normal saline. The eyeballs were then enucleated, fixed with 4% paraformaldehyde for 1 day at 4° C., dehydrated with ethanol, embedded in paraffin (Tissue-Tek TEC 5; Sakura) and sectioned into 5 μm thickness slices. The sectioned samples were then stained with cresyl violet and examined using a light microscope (Leica). All sectioned retinal samples were photographed under the same magnification (Ilford Pan-F plus film, 50 ASA).

The thickness of a retina was determined by sectioning retinal samples at the same distance (1.5 mm from the disc). To evaluate the degree of the injury caused by retinal ischemia, the whole retinal thickness [from the inner limiting membrane (ILM) to the retinal pigment epithelium (RPE) layer] and the inner retinal thickness [from the ILM to the inner nuclear layer (INL)] were measured. All the measurements were carried out by an expert who was unaware of the experimental conditions used to treat the samples.

Retrograde Fluorogold Labeling of RGCs

On the initial day, after receiving anesthesia, a 2-cm opening was made in the skin of the rat's head and two round side openings in the skull were created by a drill. These were followed by an intracranial injection of 2 μL 0.5% fluorogold (Sigma-Aldrich) through a Hamilton microsyringe at depths of 3.8, 4.0, and 4.2 mm below the level of the skull vertex. One day following the fluorogold treatment, either the pre-ischemic emodin, vehicle or Sham procedure was carried out (as described in the section administration of drug). The rats were sacrificed 3 days after the fluorogold injection was performed. After sacrifice, the retinal tissue was carefully isolated, incubated with 4% paraformaldehyde fixative, sectioned and processed. Finally, the samples were then analyzed by fluorescent microscopy. The RGC density was defined as the total RGC amount divided by the whole retinal area and its mean was then calculated.

Western Blotting

Samples of the retina were retrieved immediately after the sacrifice of the rats. Denatured proteins were then obtained using lysis buffer (mammalian protein extraction reagent; Hycell), the samples were then sonicated and quantified to give equivalent amounts of 30 μg/30 μl/well. The prepared protein samples were separated by 12% sodium dodecyl sulfate polyacrylamide gel electrophoresis (Bio-Rad, Hercules, Calif.) and then transferred to a polyvinylidene fluoride (PVDF) membrane. The PVDF membranes were incubated with blocking buffer (135 mM NaCl, 8.1 mM Na₂HPO₄, 1.5 mM KH₂PO₄, 2.7 mM KCl; pH 7.2) containing 5% fat-free skimmed milk at 4° C. for 16 hours. Next, the PVDF membranes were then incubated with various primary antibodies at 25° C. for 1 hour, there were mouse anti-β-actin monoclonal antibody (ab6276; 1:5000; Abcam Inc., Cambridge, UK), rabbit anti-ρ-catenin monoclonal antibody (ab32572; 1:5000; Abcam Inc., Cambridge, UK) and rabbit polyclonal anti-VEGF antibody (A-20; 1:200; sc-152). The blots in PVDF membranes were subsequently incubated with their appropriate secondary antibody, either horseradish peroxidase-conjugated goat anti-rabbit IgG (111-035-003; 1:2000; Jackson ImmunoResearch) or horseradish peroxidase-conjugated goat anti-mouse IgG (sc-2005; 1:2000; Santa Cruz Biotech, Santa Cruz, Calif.) at 25° C. for 1 hour. Finally, the PVDF membranes were then developed using an enhanced chemiluminescent analysis system (HyCell) and scanned using an imager (Amersham Imager; LifeSciences); the amount of each protein was quantified according to scanning densitometry.

Analysis of Statistical Significance

Unpaired Student t-tests were used to compare the statistical difference between two experimental groups. Probability (P) of <0.05 was taken to represent a statistical significant difference. All of the results are shown as mean±standard error.

Results

Cellular Experiments

Effect of Emodin on Ischemia-Induced Decrease in Cell Viability as Evaluated by MTT Assay

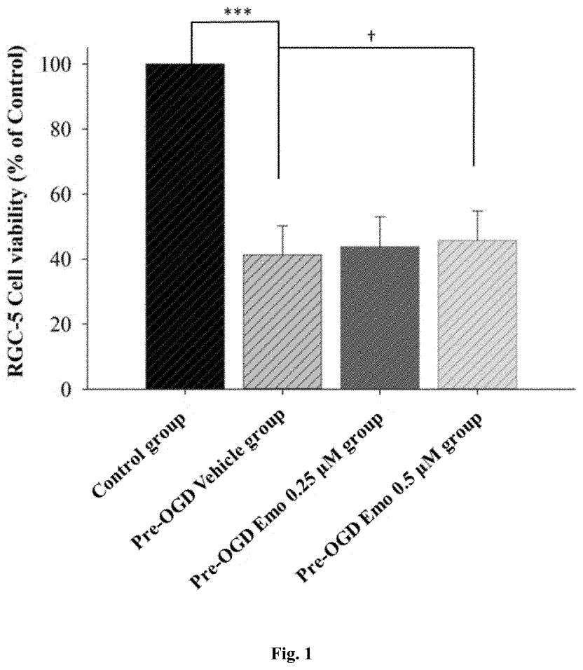

The cultured cells were used to identify the effective dose(s) of the tested agent emodin. The effect of emodin (0.25 and 0.5 μM) on the cultured cells was evaluated utilizing MTT cell viability assays and a cellular ischemia simulation model, namely OGD, for 1 day. The definition of OGD is that the cells were grown in culture medium without glucose at 37° C. under hypoxic conditions, namely 1% O₂. The results for the various experimental groups versus the control group (set as 100%; cells incubated in culture medium containing vehicle) are described subsequently (FIG. 1 and Table 1). Cells were incubated in culture medium plus one hour of pre-OGD administration of: vehicle (Pre-OGD vehicle group, cell viability of 38.30±2.51%), emodin 0.25 μM (Pre-OGD Emo 0.25 μM group, cell viability of 43.81±3.75%) and emodin 0.5 μM (Pre-OGD Emo 0.5 μM group, cell viability of 47.52±3.99%). As compared to the vehicle treatment group, emodin treatment resulted in a dosage related and significant (P=0.04; at 0.5 μM) attenuation of the OGD induced cellular injury.

Animal Experiments

Effect of Emodin on Ischemia-Induced Reduced ERG b-Wave Amplitude

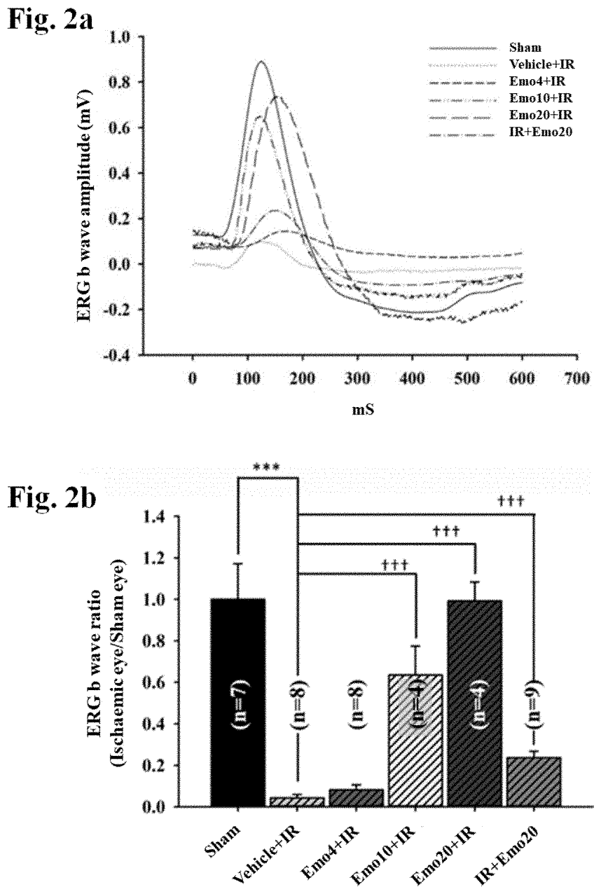

In the Sham group, the ERG b-wave amplitude/ratio was measured to be 0.87 mV/±1.00±0.00 mV (FIG. 2a; amplitude ratio of 0.82±0.14 normalized to 1 in FIG. 2b; Table 3; n=7). Following retinal ischemia, an extremely significant drop (P<0.001) in ERG b-wave amplitude/ratio was observed (FIGS. 2a/2b; Vehicle+IR=0.03 mV/0.04±0.02; n=8). Pre-ischemic intravitreous injection of emodin alleviated the ischemia induced significant decrease in ERG b-wave amplitude/ratio (FIGS. 2a/2b; Emo4+IR=0.07 mV/0.08±0.07, n=8; Emo10+IR=0.58 mV/0.64±0.28, P<0.001, n=4; Emo20+IR=0.63 mV/0.99±0.18, P<0.001, n=4). Interestingly, it was also demonstrated that post-ischemic intravitreous emodin also significantly (P<0.001) ameliorated the above ischemia induced reduction in ERG b-wave amplitude/ratio (FIGS. 2a/2b; IR+Emo20=0.12 mV/0.24±0.09, n=9). The ERG b-wave was widely believed to reflect mainly light-induced activity of bipolar cells and Müller cells. Therefore, the above ERG b-wave result showed that emodin would protect bipolar cells and Müller cells from injury caused by retinal ischemia.

Effect of Emodin on Ischemia-Induced Reduction in Cresyl Violet Stained Retina Thickness

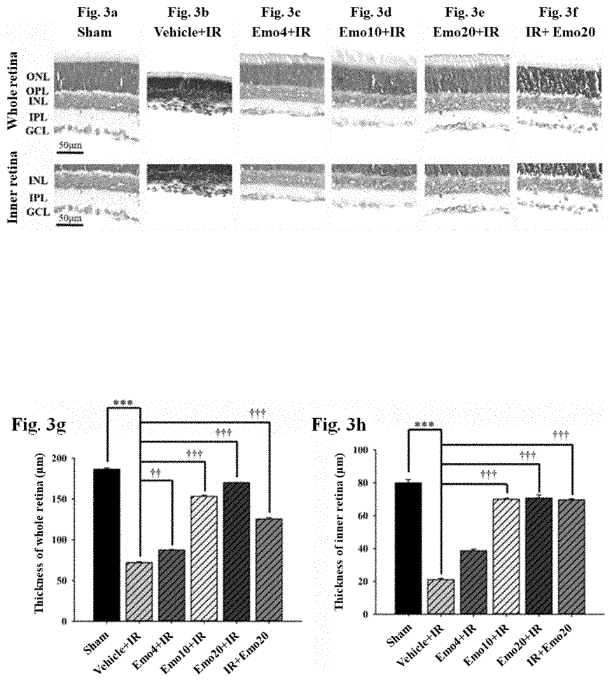

As revealed in the FIG. 3 and Table 4, in the Sham group (n=10), the retinal thickness (μm) was measured to be: whole retina (186.50±1.43) and inner retina (79.90±2.06). After induction of retinal ischemia, a significant loss of both whole retinal thickness and inner retinal thickness (Vehicle+IR: whole=71.80±1.08; inner=20.97±0.85; P<0.001) was observed. However, pre-ischemic intravitreous injection of emodin (4, 10 or 20 μM) dose-dependently (n=10; Emo4+IR: whole=87.40±0.60, inner=38.60±1.01; n=10; Emo10+IR: whole=153.20±1.48, inner=70.05±0.60; n=10; Emo20+IR: whole=170.10±0.10; inner=70.65±2.06) and significantly (P<0.01 at 4 μM; P<0.001 at 10 or 20 μM) alleviated the retinal thickness reduction caused by retinal ischemia and reperfusion. In addition, the post-ischemic intravitreous emodin (n=10; 20 μM; IR+Emo20: whole=125.45±1.68, inner=69.65±0.68) also had a significant (P<0.001) anti-ischemic effect in terms of retinal thickness.

Effect of Emodin on the Ischemia-Induced Decrease in the Retrograde Fluorogold Labeled RGC Density

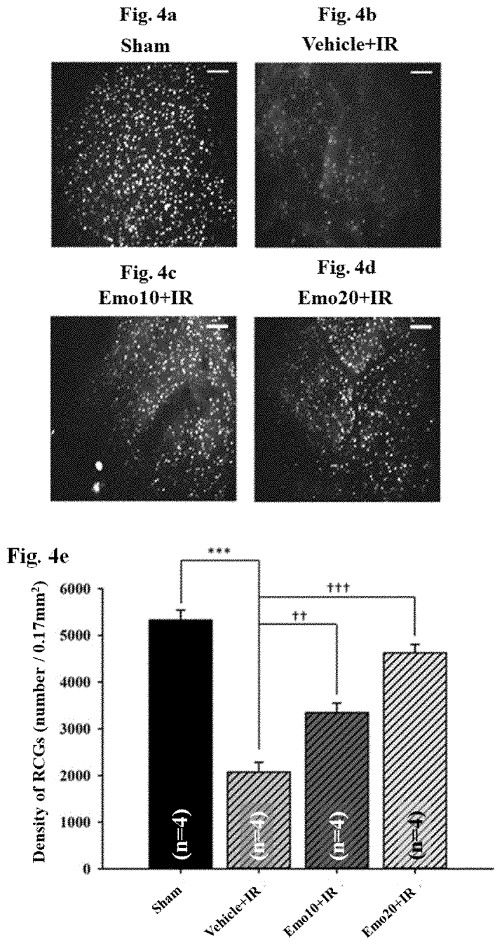

As shown in FIGS. 4a/4e and Table 5, RGC immunolabeling gave a cell density of 5323.53±215.6/0.17 mm² following the Sham procedure (Sham; n=4). An extremely significant drop (P<0.001) in RGC density was observed in the ischemic retina with pre-ischemic intravitreous injection of vehicle (n=4; Vehicle+IR=2069.12±212.82; FIGS. 4b/4e). Furthermore, there was a dose-dependent and significant (n=4; P<0.01 at 10 μM, Emo10+IR=3345.59±206.80, FIGS. 4c/4e; n=4; P<0.001 at 20 μM, Emo20+IR=4623.53±179.48, FIGS. 4d/4e) increase in the cell density after pre-ischemic administration of either 10 or 20 μM emodin.

The Effect of Emodin on the Ischemia-Induced Changes in the Protein Level Ratio of β-Catenin/VEGF to β-Actin

Western blot assays were utilized to investigate the therapeutic mechanisms by which emodin ameliorated ischemic damage. As revealed by the immunoblotting images (FIG. 5a; Table 6), pre-ischemic administration of 10 or 20 μM emodin (Emo10+IR or Emo20+IR) had a dose-dependent inhibitory effect on β-catenin/VEGF protein over-expression that was induced by ischemia compared to pre-ischemic intravitreous vehicle (Vehicle+IR). In the quantitative analysis (FIGS. 5b/5c; Table 6), the effects of emodin on β-catenin/VEGF protein levels were calculated using the ratio of β-catenin/VEGF:β-actin with the Sham group (n=6) normalized to 1. After an ischemic insult, it was observed that there was a dose-dependent (less effect at 10 μM; Emo10+IR=1.18±0.24/3.99±2.86; n=6) and significant effect (P=0.02/0.03 at 20 μM; Emo20+IR=1.00±0.19/1.23±0.44; n=6) of emodin whereby it blunted the significant (P<0.001) upregulation of β-catenin/VEGF protein that was brought about by ischemia compared to pre-ischemic intravitreous vehicle (Vehicle+IR=1.64±0.14/7.67±2.57; n=6). Results that were presented in brackets follow the format of β-catenin/VEGF.

One skilled in the art readily appreciates that the present invention is well adapted to carry out the objects and obtain the ends and advantages mentioned, as well as those inherent therein. The methods and uses thereof are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method for treating a subject suffering from retinal ischemia, or a disease, condition, or disorder associated with retinal ischemia, comprising administering to the subject a therapeutically effective amount of emodin or a composition comprising emodin.

2. The method of claim 1, wherein the disease, condition, or disorder associated with retinal ischemia comprises retinal vascular occlusion, glaucomatous optic neuropathy, proliferative diabetic retinopathy, neovascular age-related macular degeneration, familial exudative vitreoretinopathy (FEVR), Coats' disease, persistent hyperplastic primary vitreous (PHPV), Norrie disease or retinopathy of prematurity (ROP).

3. The method of claim 1, wherein the subject is a mammal.

4. The method of claim 1, wherein the subject is a human.

5. The method of claim 1, which further comprises administering to the subject a pharmaceutically acceptable adjuvant, vehicle, or carrier.

6. The method of claim 1, wherein the therapeutically effective amount of emodin or the composition comprising emodin ranges from 2 to 30 μM.

7. The method of claim 1, wherein the therapeutically effective amount of emodin or the composition comprising emodin ranges from 4 to 20 μM.
